(19) United States
(12) Patent Application Publication
(10) Pub. No.: US 2025/0372240 A1
(43) Pub. Date: Dec. 4, 2025

(54) SYSTEM AND METHOD FOR AI-BASED UNIVERSAL HEALTHCARE PLATFORM

(71) Applicant: Lennie Furr, Hixson, TN (US)

(72) Inventor: Lennie Furr, Hixson, TN (US)

(21) Appl. No.: 18/680,240

(22) Filed: May 31, 2024

(51) Int. Cl. G16H 40/20; G16H 10/60; G16H 50/70

(52) U.S. Cl. CPC G16H 40/20; G16H 10/60; G16H 50/70

(57) ABSTRACT

A system for an automated medical data processing based on patient-related data, including a processor of a healthcare processing server node configured to host at least Artificial Intelligence (AI) and machine learning (ML) modules and connected to at least one medical records cloud-based database and to at least one patient entity node over a network; and a memory on which are stored machine-readable instructions that when executed by the processor, cause the processor to: acquire user account creation and input data from at least one patient entity node using an OCR module; analyze patient intake data derived from the input data by an AI module configured to analyze the intake data; process user insurance data by an insurance AI module configured to generate an insurance verification verdict; acquire recommended lab test and triage data of the user and ingest the lab test and the triage data into an AI module configured to analyze the lab tests; receive treatment and medication suggestions and generate a feature vector based on the treatment and medication suggestions; provide the feature vector to an ML module configured to generate at least one clinical outcome model; derive clinical documentation data from the patient intake data and from the at least one medical records cloud-based database and apply NPL processing to the clinical documentation data; and acquire revenue cycle data from the clinical documentation data and ingest the revenue cycle data into an AI module configured to generate billing parameters.

### FIELD OF DISCLOSURE

The present disclosure generally relates to healthcare automation, and more particularly, to an AI-based healthcare automated system for real-time universal healthcare platform processing based on predictive analytics of patients'-related data and stored historical heuristic data.

### BACKGROUND

Multitude of healthcare-relate management platforms are used by medical facilities worldwide. Most of the existing platforms use Manual Patient Intake and Record-Keeping. These processes are time-consuming, prone to human error, inefficient in managing large volumes of patient data, and challenging to retrieve specific patient information quickly. These in turn pose increased administrative burden on healthcare staff, create potential for data inaccuracies and security risks due to physical records.

Existing Electronic Healthcare Record systems (EHRs) have limited interoperability between different healthcare systems, user interface complexities, and insufficient customization options for specific practice needs. These systems experience hindered workflow efficiency, increased training requirements for staff, and potential resistance to adoption due to usability issues.

In addition to the EHRs, Standalone Medical Billing Software is commonly used. These applications may have lack of integration with clinical workflows and EHR systems, leading to duplicated efforts and discrepancies in patient data. These applications also have increased chances of billing errors, delayed reimbursements, and challenges in revenue cycle management.

Separate Patient Portal Solutions are also used. These solutions have limited functionalities, often restricted to appointment scheduling and basic communication, without comprehensive access to health records or personalized health insights. As such, these solutions may cause missed opportunities for enhancing patient engagement, limited patient empowerment, and disjointed patient experience.

Conventional Decision Support Systems are limited based on predefined rules without the capability to learn from new data, resulting in outdated recommendations over time. Furthermore, these systems have limited adaptability to evolving medical knowledge, inability to provide personalized treatment recommendations, and potential for outdated clinical guidance.

In summary, the above listed conventional solutions and methods, while foundational in the transition from paper-based to digital healthcare systems, often fall short in delivering the efficiency, accuracy, and patient-centered care now achievable with modern Healthcare Automation systems. However, the Healthcare Automation system use large data analytics that causes high resource usage and associated high costs.

Accordingly, a system and method for AI-based healthcare automated system for real-time universal healthcare platform processing based on predictive analytics of patients'-related data and stored historical heuristic data are desired.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

One embodiment of the present disclosure provides a system for an automated medical data processing based on patient-related data, including a processor of a healthcare processing server node configured to host at least Artificial Intelligence (AI) and machine learning (ML) modules and connected to at least one medical records cloud-based database and to at least one patient entity node over a network; and a memory on which are stored machine-readable instructions that when executed by the processor, cause the processor to: acquire user account creation and input data from at least one patient entity node using an OCR module; analyze patient intake data derived from the input data by an AI module configured to analyze the intake data; process user insurance data by an insurance AI module configured to generate an insurance verification verdict; acquire recommended lab test and triage data of the user and ingest the lab test and the triage data into an AI module configured to analyze the lab tests; receive treatment and medication suggestions and generate a feature vector based on the treatment and medication suggestions; provide the feature vector to an ML module configured to generate at least one clinical outcome model; derive clinical documentation data from the patient intake data and from the at least one medical records cloud-based database and apply NPL processing to the clinical documentation data; and acquire revenue cycle data from the clinical documentation data and ingest the revenue cycle data into an AI module configured to generate billing parameters.

Another embodiment of the present disclosure provides a method that includes one or more of: acquiring user account creation and input data from at least one patient entity node using an OCR module; analyzing patient intake data derived from the input data by an AI module configured to analyze the intake data; processing user insurance data by an insurance AI module configured to generate an insurance verification verdict; acquiring recommended lab test and triage data of the user and ingesting the lab test and the triage data into an AI module configured to analyze the lab tests; receiving treatment and medication suggestions and generating a feature vector based on the treatment and medication suggestions; providing the feature vector to an ML module configured to generate at least one clinical outcome model; deriving clinical documentation data from the patient intake data and from the at least one medical records cloud-based database and applying NPL processing to the clinical documentation data; and acquiring revenue cycle data from the clinical documentation data and ingesting the revenue cycle data into an AI module configured to generate billing parameters.

Another embodiment of the present disclosure provides a computer-readable medium including instructions for acquiring user account creation and input data from at least one patient entity node using an OCR module; analyzing patient intake data derived from the input data by an AI module configured to analyze the intake data; processing user insurance data by an insurance AI module configured to generate an insurance verification verdict; acquiring recommended lab test and triage data of the user and ingesting the lab test and the triage data into an AI module configured to analyze the lab tests; receiving treatment and medication suggestions and generating a feature vector based on the treatment and medication suggestions; providing the feature vector to an ML module configured to generate at least one clinical outcome model; deriving clinical documentation data from the patient intake data and from the at least one medical records cloud-based database and applying NPL processing to the clinical documentation data; and acquiring revenue cycle data from the clinical documentation data and ingesting the revenue cycle data into an AI module configured to generate billing parameters.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a network diagram of a system for AI-based healthcare automated system for real-time universal healthcare platform, consistent with the present disclosure;

FIG. 2 illustrates a network diagram of a system including detailed features of a Healthcare Processing Server (HPS) node consistent with the present disclosure;

FIG. 3A illustrates a flowchart of a method for AI-based healthcare automated system for real-time universal healthcare platform processing based on predictive analytics of patients'-related data and stored historical heuristic data consistent with the present disclosure;

FIG. 3B illustrates a further flow chart of a method for AI-based healthcare automated system for real-time universal healthcare platform processing based on predictive analytics of patients'-related data and stored historical heuristic data consistent with the present disclosure;

FIG. 4 illustrates deployment of a machine learning model for prediction of healthcare-related predictive parameters using stored data sets consistent with the present disclosure;

FIG. 5 illustrates a block diagram of a system including a computing device for performing the method of FIGS. 3A and 3B.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of processing job applicants, embodiments of the present disclosure are not limited to use only in this context.

The present disclosure provides a system, method and computer-readable medium for an AI-based healthcare automated system for real-time universal healthcare platform processing based on predictive analytics of patients'-related data and stored historical heuristic data.

The disclosed embodiments employ AI and machine learning, comprehensive data analytics, seamless integration across healthcare ecosystems, and patient empowerment tools. The healthcare universal platform, according to the disclosed embodiments, represents significant advancements over the conventional methods and systems, addressing many of their inherent limitations and disadvantages discussed in the backgrounds section of this application.

In one embodiment of the present disclosure, the system provides for AI and machine learning (ML)-generated various healthcare parameters to be used for analysis and generation of multitude of patient-related notifications. In one embodiment, an automated decision model may be generated to provide for procedure parameters associated with a patients' current status, past procedure-related behavior based on previous doctors' feedback, medical reports, social media accounts of the patient, etc. The automated notification decision model may use historical patients' data collected at the current locations (i.e., a hospital or other medical facility site) and at other remote medical facilities of the same type located within a certain range from the current location or even located globally. The relevant patients' data may include data related to other patients having the same parameters such as diagnosis, age, race, gender, language, preferred treatment conditions or locations, etc.

In one disclosed embodiment, the AI/ML technology may be combined with a data security technology for secure use of the patients'-related data. The disclosed embodiment may produce a detailed safety or success rates score on the successful treatment or therapy likelihood for the given patient based on collected patients' behavioral data. This allows for direct reporting on a trust level of the given patient to the medical entities (i.e., doctors, hospitals, emergency services, etc.).

In one embodiment, the disclosed system relates to a SaaS platform that matches patients with healthcare providers. The disclosed healthcare platform may match potential patients with doctors based on the predictive parameters that are processed through an AI machine-learning module that may automatically choose the doctor or facility that best fit the patients' requirements most accurately. Patients can receive AI-generated recommendations for providers based on their insurance and requirements. The matching module may automatically choose the doctors(s) or facilities that best fits the patient's parameters derived from the initial patient intake data.

The disclosed process, advantageously, eliminates the need for manual data analytics or automated big data analytics by processing patients' data directly on a granular level based on the AI-based predictive analysis and recommendations. This process includes transparent patient treatment mechanism coupled with a secure communications AI-based chat channel which supports multiple parties within a healthcare system.

In one embodiment, the platform implements Automated Patient Intake. The platform uses Optical Character Recognition (OCR) to automatically capture and digitize patient information from IDs and insurance cards at the point of care. AI algorithms then process this information to pre-fill forms and patient records, reducing manual data entry and the potential for errors.

In another embodiment, the platform implements Insurance Verification and Billing Automation by utilizing integrated APIs with insurance providers and clearinghouses. The system automatically verifies patient coverage and streamlines the billing process, from claim submission to payment reconciliation. The platform provides Treatment and Medication Suggestions by leveraging AI and machine learning. The platform application analyzes patient data, including medical history and current symptoms, to suggest personalized treatment plans and medication recommendations, aiding clinicians in decision-making. The platform may provide for Patient Engagement and Education. Through a patient portal application, patients can access their health records, manage appointments, and receive personalized health insights generated by the system, promoting patient engagement and empowerment.

In one embodiment, Real-Time Data Analytics are provided offering healthcare providers actionable insights into patient health trends, treatment outcomes, and operational metrics, enabling data-driven decisions to improve care quality and practice efficiency. The disclosed universal healthcare platform provides for Security and Compliance by implementing robust security measures to protect sensitive patient data and ensure compliance with healthcare regulations, such as HIPAA, through encryption, secure data storage, and controlled access mechanisms. The universal healthcare platform provides for Interoperability-i.e., facilitates seamless data exchange between different healthcare systems and devices, enhancing care coordination and ensuring comprehensive patient records are easily accessible to authorized healthcare providers.

The disclosed universal healthcare platform may be used by doctors, nurses, and administrative staff to enhance patient care delivery, improve workflow efficiency, and reduce administrative burdens, allowing more time to be dedicated to patient care. The platform may allow patients to actively participate in their healthcare through easy access to their health information, direct communication with healthcare providers, and tools to manage their health and wellness. The platform may help healthcare administrators monitor and improve operational aspects of healthcare delivery, including patient flow, staff allocation, and financial management, through comprehensive analytics and reporting tools. By integrating AI-based automation technologies, the disclosed universal healthcare platform aims to address the challenges of modern healthcare systems, offering a comprehensive solution that improves efficiency, enhances patient care, and reduces operational costs.

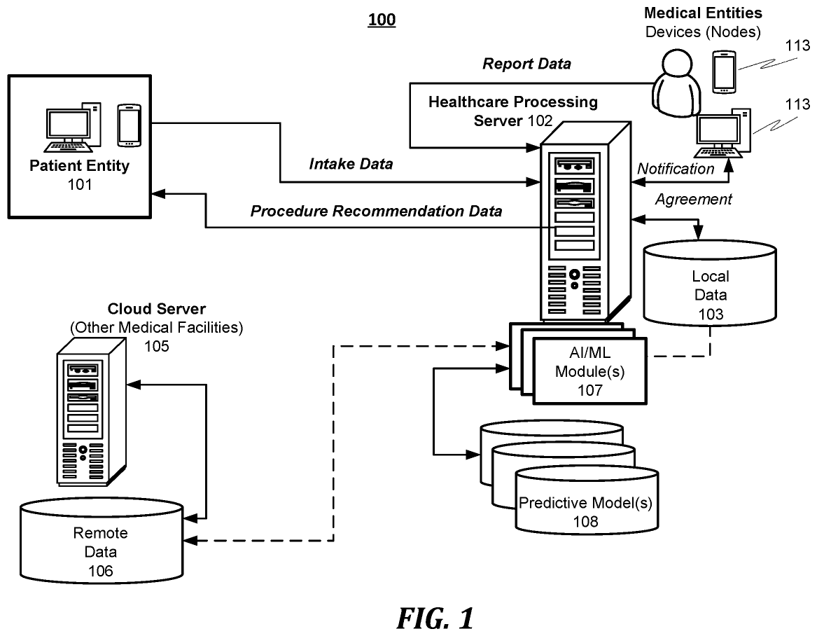

FIG. 1 illustrates a network diagram of a system for AI-based healthcare automated universal healthcare platform processing based on predictive analytics of patients'-related data and stored historical heuristic data consistent with the present disclosure.

Referring to FIG. 1, the example network 100 includes the Healthcare Processing Server (HPS) node 102 connected to a cloud server node(s) 105 over a network. The HPS node 102 is configured to host AI/ML module(s) 107. The HPS node 102 may receive patient intake data from a patient entity 101. The employment request data may have hashtags representing the employment parameters. In one embodiment, the patient intake data may be processed by the HPS node 102 to parse out the key features that may be used for building classifiers/feature vectors to be ingested by the AI/ML module(s) 107.

The HPS node 102 may query a local patients' database for the historical local patients' data 103 associated with the current patient intake data. The HPS node 102 may acquire relevant remote patients' data 106 from a remote database residing on a cloud server 105. The remote patients' data 106 may be collected from other medical facilities. The remote patients' data 106 may be collected from patients that had the same (or similar) diagnosis, age, gender, race, language, etc. as the local patients' who are associated with the current patient intake data.

As discussed above, the HPS node 102 may generate a feature vector or classifier based on the patient intake data and the collected patients' data (i.e., pre-stored local data 103 and remote data 106). The HPS node 102 may ingest the feature vector into AI/ML module(s) 107. The AI/ML module(s) 107 may generate predictive model(s) 108 based on the feature vector data to predict various universal healthcare platform parameters for automatically generating notification(s) to be provided to patients associated with entities 101 and medical entities 113 (e.g., nurses, doctor(s), care providers, managers, etc.). The healthcare parameters may be further analyzed by the HPS node 102 prior to generation of the notification(s). In one embodiment, the healthcare parameters may be used for adjustment of the treatment, therapy, and/or schedule based on availability of the selected (i.e., matched) practitioners or facilities.

The AI/ML module(s) 107 may generate predictive model(s) 108 to predict the healthcare-related parameters for the patients in response to the specific relevant pre-stored patients'-related data acquired from the database 103. This way, the current predictive healthcare parameters may be predicted based not only on the current patient-related data (e.g., intake data) and current other healthcare-related data, but also based on the previously collected patients' heuristics and healthcare-related data associated with the given patient data or current parameters derived from the heuristics data stored on a central database (not shown).

Regarding the patient data security within the disclosed AI-based healthcare automated universal healthcare platform 100, the following security measures may be implemented.

Secure user/patient authentication may be implemented using a Hitrust™ compliant service like Firebase authentication (under Google Cloud). The system may also have login access via email one-time sign-in link and phone one-time-password options. Access control may be implemented as follows. The system may be accessible to the users based on roles and permissions. Based on the role, the backend APIs and frontend features will be accessible from a secured login area. In one embodiment, the Protected Health Information (PHI) may be stored in the encrypted form in database. In one embodiment, MongoDB™ data encryption may be used to employ its robust features to protect healthcare data while in-transit (network), at-rest (storage), and in-use (memory, logs). Customers can use automatic encryption of key data fields like PII, PHI, or any data deemed sensitive—ensuring data is encrypted throughout its lifecycle.

Regarding secure connections, a Virtual Private Cloud (VPC) setup may be used to connect to the database securely. Note that only the backend will be authorized to access the database. The Encrypt Data in Transit may be addressed by having data transit from the API to the database being encrypted. MongoDB Atlas™ supports SSL/TLS by default. The NodeJS application may be configured to use TLS/SSL by specifying the SSL options in the connection string or as part of the MongoClient options.

According to the exemplary embodiments, all information is exchanged over secured API connections using HTTPS. In one embodiment, an automatic session timeout after x(=15) minutes of inactivity may need to be implemented. S3 storage encryption and access control may be implemented as well. For all S3 document uploads, the system may have enabled “Encryption at Rest: Use server-side encryption (SSE)” to encrypt all patient related documents. The system may use SSE-KMS: AWS Key Management Service (SSE-KMS). Identity and Access Management (IAM) policies may be used to restrict access to S3 buckets and objects. The external third-party service used (like the AWS services) are HIPAA compliant within the system. The system may use comprehensive audit trails to track and log all activities within the system that are maintained. This is crucial for compliance and accountability.

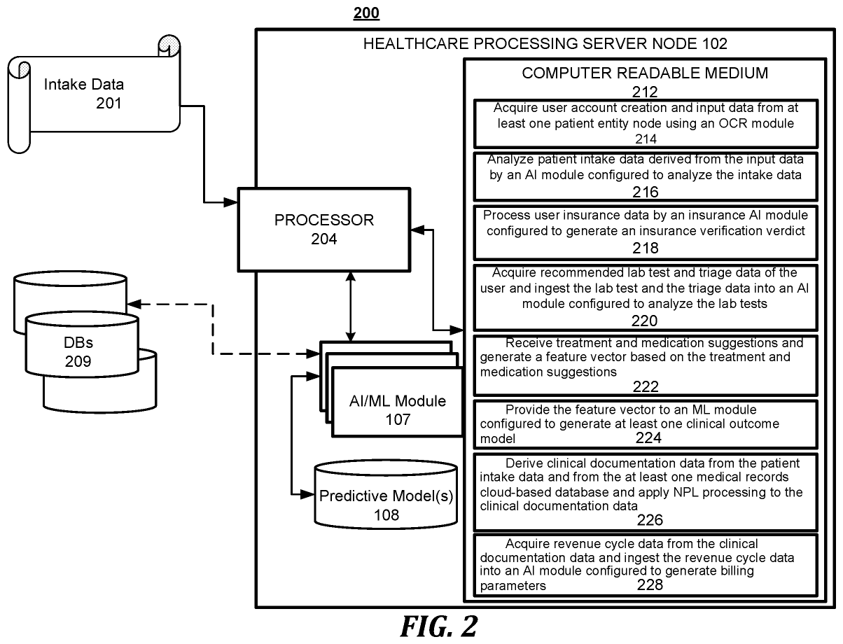

FIG. 2 illustrates a network diagram of a system including detailed features of a Healthcare Processing Server (HPS) node consistent with the present disclosure.

Referring to FIG. 2, the example network 200 includes the HPS node 102 connected to medical entities device(s) 113 and patient entities 101 to receive patient intake data 201. The HPS node 102 is configured to host AI/ML module(s) 107. As discussed above with respect to FIGS. 1, the HPS node 102 may receive patient intake data 201 provided by the patient entities 101 (FIG. 1) and pre-stored patients' data retrieved from local and remote databases. As discussed above, the pre-stored patients' data may be retrieved from the central database(s) 209.

The AI/ML module(s) 107 may generate predictive model(s) 108 based on the received patient intake data 201 and the patients'-related data provided by the HPS node 102. As discussed above, the AI/ML modules 107 may provide predictive outputs data in a form of healthcare parameters for automatic generation of reports, diagnoses, insights, notifications, etc. for multiple parties within the healthcare system. The HPS node 102 may process the predictive outputs data received from the AI/ML module(s) 107 to generate the notification of a current risk assessment ranking pertaining to a particular patient or patient-related procedure or therapy.

In one embodiment, the HPS node 102 may acquire patient records (or intake data) data periodically in order to check if new healthcare parameters for automatic generation of reports, diagnoses, insights, notifications need to be generated or the treatment schedule needs to be reset. In another embodiment, the HPS node 102 may continually monitor patients'-related data acquired from databases 209 and may detect a parameter that deviates from a previous recorded parameter (or from a median reading value) by a margin that exceeds a threshold value pre-set for this particular parameter. For example, if a patient's lab tests change this may cause a drastic change in this patient's healthcare parameters pertaining to treatment or treatment schedule. As another non-limiting example, a change in patient's insurance may also cause critical changes in treatment possibilities or options. Accordingly, once the threshold is met or exceeded by at least one healthcare parameter of the patient, the HPS node 102 may provide the currently acquired patient's parameter to the AI/ML module(s) 107 to generate a list of updated healthcare parameters based on the current patient's conditions and medical requirements.

While this example describes in detail only one HPS node 102, multiple such nodes may be connected to the network and to the databases 209. It should be understood that the HPS node 102 may include additional components and that some of the components described herein may be removed and/or modified without departing from a scope of the HPS node 102 disclosed herein. The HPS node 102 may be a computing device or a server computer, or the like, and may include a processor 204, which may be a semiconductor-based microprocessor, a central processing unit (CPU), an application specific integrated circuit (ASIC), a field-programmable gate array (FPGA), and/or another hardware device. Although a single processor 204 is depicted, it should be understood that the HPS node 102 may include multiple processors, multiple cores, or the like, without departing from the scope of the HPS node 102 system.

The HPS node 102 may also include a non-transitory computer readable medium 212 that may have stored thereon machine-readable instructions executable by the processor 204. Examples of the machine-readable instructions are shown as 214-222 and are further discussed below. Examples of the non-transitory computer readable medium 212 may include an electronic, magnetic, optical, or other physical storage device that contains or stores executable instructions. For example, the non-transitory computer readable medium 212 may be a Random-Access memory (RAM), an Electrically Erasable Programmable Read-Only Memory (EEPROM), a hard disk, an optical disc, or other type of storage device.

The processor 204 may fetch, decode, and execute the machine-readable instructions 214 to acquire user account creation and input data from at least one patient entity node using an OCR module. The processor 204 may fetch, decode, and execute the machine-readable instructions 216 to analyze patient intake data derived from the input data by an AI module configured to analyze the intake data. The processor 204 may fetch, decode, and execute the machine-readable instructions 218 to process user insurance data by an insurance AI module configured to generate an insurance verification verdict. The processor 204 may fetch, decode, and execute the machine-readable instructions 220 to acquire recommended lab test and triage data of the user and ingest the lab test and the triage data into an AI module configured to analyze the lab tests.

The processor 204 may fetch, decode, and execute the machine-readable instructions 222 to receive treatment and medication suggestions and generate a feature vector based on the treatment and medication suggestions. The processor 204 may fetch, decode, and execute the machine-readable instructions 224 to provide the feature vector to an ML module configured to generate at least one clinical outcome model. The processor 204 may fetch, decode, and execute the machine-readable instructions 226 to derive clinical documentation data from the patient intake data and from the at least one medical records cloud-based database and apply NPL processing to the clinical documentation data. The processor 204 may fetch, decode, and execute the machine-readable instructions 228 to acquire revenue cycle data from the clinical documentation data and ingest the revenue cycle data into an AI module configured to generate billing parameters.

The central database(s) 209 may be configured to use one or more APIs that manage transactions for multiple participating nodes and for recording the transactions on the central database(s) 209.

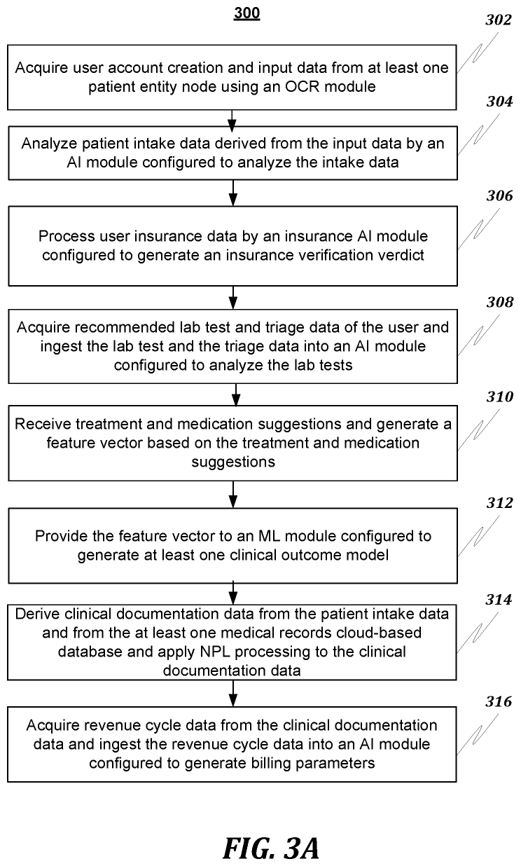

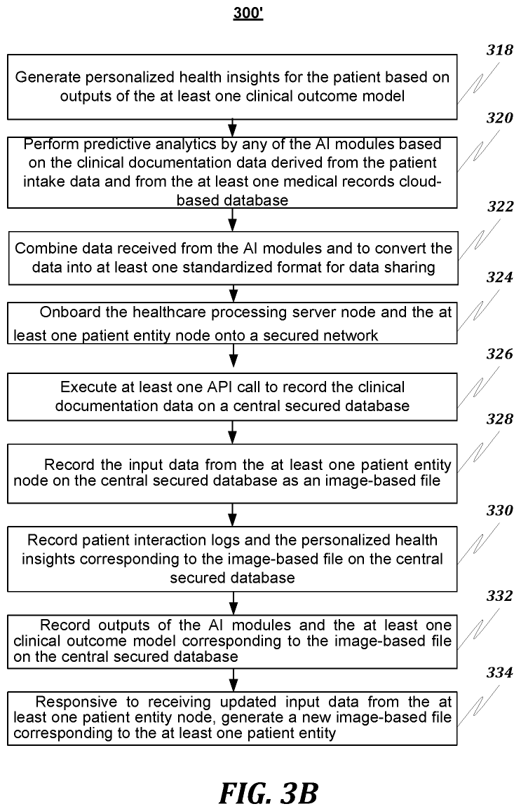

FIG. 3A illustrates a flowchart of a method for an AI-based healthcare automated universal healthcare platform processing based on predictive analytics of patients'-related data and stored historical heuristic data consistent with the present disclosure.

Referring to FIG. 3A, the method 300 may include one or more of the steps described below. FIG. 3A illustrates a flow chart of an example method executed by the HPS 102 (see FIG. 2). It should be understood that method 300 depicted in FIG. 3A may include additional operations and that some of the operations described therein may be removed and/or modified without departing from the scope of the method 300. The description of the method 300 is also made with reference to the features depicted in FIG. 2 for purposes of illustration. Particularly, the processor 204 of the HPS node 102 may execute some or all of the operations included in the method 300.

With reference to FIG. 3A, at block 302, the processor 204 may acquire user account creation and input data from at least one patient entity node using an OCR module. At block 304, the processor 204 may analyze patient intake data derived from the input data by an AI module configured to analyze the intake data. At block 306, the processor 204 may process user insurance data by an insurance AI module configured to generate an insurance verification verdict. At block 308, the processor 204 may acquire recommended lab test and triage data of the user and ingest the lab test and the triage data into an AI module configured to analyze the lab tests. At block 310, the processor 204 may receive treatment and medication suggestions and generate a feature vector based on the treatment and medication suggestions. At block 312, the processor 204 may provide the feature vector to an ML module configured to generate at least one clinical outcome model. At block 314, the processor 204 may derive clinical documentation data from the patient intake data and from the at least one medical records cloud-based database and apply NPL processing to the clinical documentation data.

At block 316, the processor 204 may acquire revenue cycle data from the clinical documentation data and ingest the revenue cycle data into an AI module configured to generate billing parameters.

FIG. 3B illustrates a further flow chart of a method for an AI-based healthcare automated universal healthcare platform processing based on predictive analytics of patients'-related data and stored historical heuristic data consistent with the present disclosure.

Referring to FIG. 3B, the method 300′ may include one or more of the steps described below. FIG. 3B illustrates a flow chart of an example method executed by the HPS 102 (see FIG. 2). It should be understood that method 300′ depicted in FIG. 3B may include additional operations and that some of the operations described therein may be removed and/or modified without departing from the scope of the method 300′. The description of the method 300′ is also made with reference to the features depicted in FIG. 2 for purposes of illustration. Particularly, the processor 204 of the HPS 102 may execute some or all of the operations included in the method 300′.

With reference to FIG. 3B, at block 318, the processor 204 may generate personalized health insights for the patient based on outputs of the at least one clinical outcome model. At block 320, the processor 204 may perform predictive analytics by any of the AI modules based on the clinical documentation data derived from the patient intake data and from the at least one medical records cloud-based database. At block 322, the processor 204 may combine data received from the AI modules and to convert the data into at least one standardized format for data sharing. At block 324, the processor 204 may onboard the healthcare processing server node and the at least one patient entity node onto a secured network.

At block 326, the processor 204 may execute at least one API call to record the clinical documentation data on a central secured database. At block 328, the processor 204 may record the input data from the at least one patient entity node on the central secured database as an image-based file.

At block 330, the processor 204 may record patient interaction logs and the personalized health insights corresponding to the image-based file on the central secured database. At block 332, the processor 204 may record outputs of the AI modules and the at least one clinical outcome model corresponding to the image-based file on the central secured database. At block 334, the processor 204 may responsive to receiving updated input data from the at least one patient entity node, generate a new image-based file corresponding to the at least one patient entity.

In one disclosed embodiment, the healthcare parameters' model(s) may be generated by the AI/ML module(s) 107 that may use training data sets to improve accuracy of the prediction of the parameters for the patient entities 101 (FIG. 1). The parameters used in training data sets may be stored in a centralized local database (such as one used for storing local patients' data 103 depicted in FIG. 1). In one embodiment, a neural network may be used in the AI/ML module 107 for healthcare parameters modeling and predictions.

In another embodiment, the AI/ML module 107 may use a central storage such as a database(s) 209 (see FIG. 2) that is a distributed storage system, which includes multiple nodes that communicate with each other.

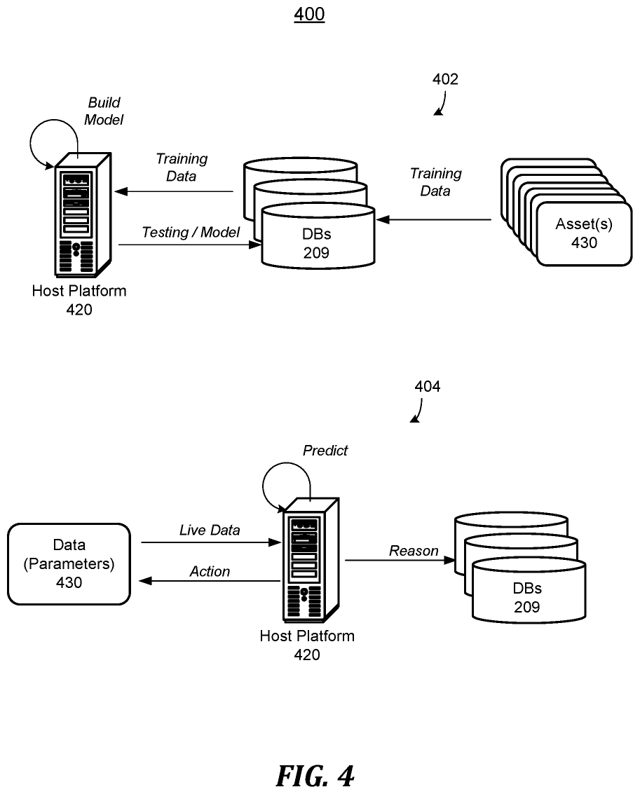

In the example depicted in FIG. 4, a host platform 420 (such as the HPS node 102) builds and deploys a machine learning model for predictive monitoring of data sets 430. Here, the host platform 420 may be a cloud platform, an industrial server, a web server, a personal computer, a user device, and the like. The data sets 430 can represent notifications or healthcare parameters. The databases 209 can be used to significantly improve both a training process 402 of the machine learning model and the healthcare parameters' predictive process 405 based on a trained machine learning model. For example, in 402, rather than requiring a data scientist/engineer or other user to collect the data, historical data (heuristics—i.e., patients'-related data) may be stored by the data sets 430 themselves (or through an intermediary, not shown) on the databases 209.

This can significantly reduce the collection time needed by the host platform 420 when performing predictive model training. For example, using API calls, data can be directly and reliably transferred straight from its place of origin, e.g., from the patient entities or from medical entities database to the central databases 209. By using the central databases 209 to ensure the security and ownership of the collected data, the APIs may directly send the data from the data sets to the entities that use the data for building a machine learning model. This allows for sharing of data among the data sets 430. The collected data may be stored in the central databases 209 based on a consensus among the entities. The consensus mechanism may be used by the APIs to ensure that the data being recorded is verified and accurate. The data recorded may be time-stamped and cryptographically signed. It is therefore auditable, transparent, and secure.

Furthermore, training of the machine learning model on the collected data may take rounds of refinement and testing by the host platform 420. Each round may be based on additional data or data that was not previously considered to help expand the knowledge of the machine learning model. In 402, the different training and testing steps (and the data associated therewith) may be stored on the central databases 209 by the host platform 420. Each refinement of the machine learning model (e.g., changes in variables, weights, etc.) may be stored on the databases 209. This provides verifiable proof of how the model was trained and what data was used to train the model. Furthermore, when the host platform 420 has achieved a finally trained model, the resulting model itself may be stored on the databases 209.

After the model has been trained, it may be deployed to a live environment where it can make employment-related predictions/decisions based on the execution of the final trained machine learning model using the healthcare parameters. In this example, data fed back from the asset 430 may be input into the machine learning model and may be used to make event predictions such as most optimal patient treatment, medical facilities and scheduling parameters for treatment, etc. Determinations made by the execution of the machine learning model (e.g., notification or treatment scheduling parameters, etc.) at the host platform 420 may be stored on the databases 209 to provide auditable/verifiable proof. As one non-limiting example, the machine learning model may predict a future change of a part of the data set 430 (the alert parameters-i.e., assessment of risk of treatment, etc.). The data behind this decision may be stored by the host platform 420 on the databases 209.

The above embodiments of the present disclosure may be implemented in hardware, in a computer-readable instructions executed by a processor, in firmware, or in a combination of the above. The computer computer-readable instructions may be embodied on a computer-readable medium, such as a storage medium. For example, the computer computer-readable instructions may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

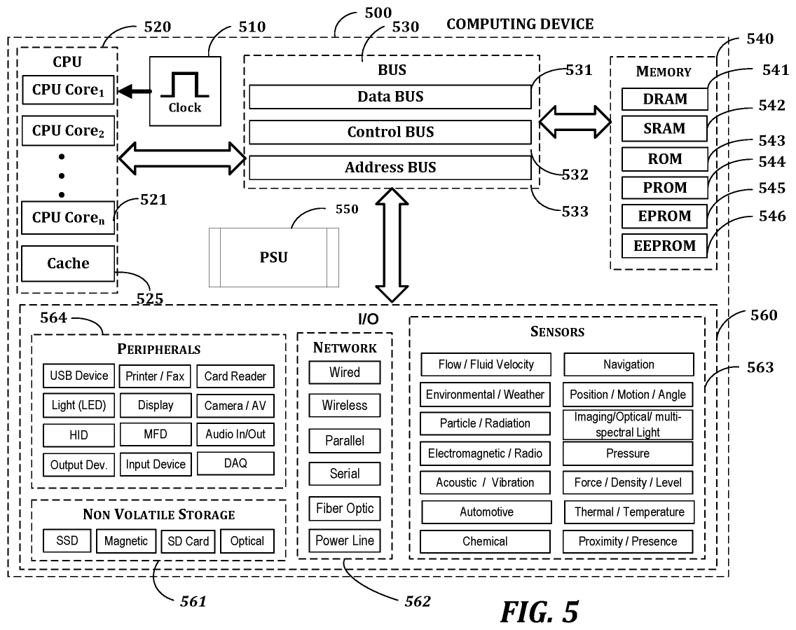

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative embodiment, the processor and the storage medium may reside as discrete components. For example, FIG. 5 illustrates an example computing device (e.g., a server node) 500, which may represent or be integrated in any of the above-described components, etc.

FIG. 5 illustrates a block diagram of a system including computing device 500. The computing device 500 may comprise, but not be limited to the following:
Mobile computing device, such as, but is not limited to, a laptop, a tablet, a smartphone, a drone, a wearable, an embedded device, a handheld device, an Arduino, an industrial device, or a remotely operable recording device;
A supercomputer, an exa-scale supercomputer, a mainframe, or a quantum computer;
A minicomputer, wherein the minicomputer computing device comprises, but is not limited to, an IBM AS500/iSeries/System I, A DEC VAX/PDP, a HP3000, a Honeywell-Bull DPS, a Texas Instruments TI-990, or a Wang Laboratories VS Series;
A microcomputer, wherein the microcomputer computing device comprises, but is not limited to, a server, wherein a server may be rack mounted, a workstation, an industrial device, a raspberry pi, a desktop, or an embedded device;

The HPS node 102 (see FIG. 2) may be hosted on a centralized server or on a cloud computing service. Although method 300 has been described to be performed by the HPS node 102 implemented on a computing device 500, it should be understood that, in some embodiments, different operations may be performed by a plurality of the computing devices 500 in operative communication at least one network.

Embodiments of the present disclosure may comprise a computing device having a central processing unit (CPU) 520, a bus 530, a memory unit 550, a power supply unit (PSU) 550, and one or more Input/Output (I/O) units. The CPU 520 coupled to the memory unit 550 and the plurality of I/O units 560 via the bus 530, all of which are powered by the PSU 550. It should be understood that, in some embodiments, each disclosed unit may actually be a plurality of such units for the purposes of redundancy, high availability, and/or performance. The combination of the presently disclosed units is configured to perform the stages any method disclosed herein.

Consistent with an embodiment of the disclosure, the aforementioned CPU 520, the bus 530, the memory unit 550, a PSU 550, and the plurality of I/O units 560 may be implemented in a computing device, such as computing device 500. Any suitable combination of hardware, software, or firmware may be used to implement the aforementioned units. For example, the CPU 520, the bus 530, and the memory unit 550 may be implemented with computing device 500 or any of other computing devices 500, in combination with computing device 500. The aforementioned system, device, and components are examples and other systems, devices, and components may comprise the aforementioned CPU 520, the bus 530, the memory unit 550, consistent with embodiments of the disclosure.

At least one computing device 500 may be embodied as any of the computing elements illustrated in all of the attached figures, including the design server node 102 (FIG. 2). A computing device 500 does not need to be electronic, nor even have a CPU 520, nor bus 530, nor memory unit 550. The definition of the computing device 500 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 500, especially if the processing is purposeful.

With reference to FIG. 5, a system consistent with an embodiment of the disclosure may include a computing device, such as computing device 500. In a basic configuration, computing device 500 may include at least one clock module 510, at least one CPU 520, at least one bus 530, and at least one memory unit 550, at least one PSU 550, and at least one I/0 560 module, wherein I/O module may be comprised of, but not limited to a non-volatile storage sub-module 561, a communication sub-module 562, a sensors sub- module 563, and a peripherals sub-module 565.

A system consistent with an embodiment of the disclosure the computing device 500 may include the clock module 510 may be known to a person having ordinary skill in the art as a clock generator, which produces clock signals. Clock signal is a particular type of signal that oscillates between a high and a low state and is used like a metronome to coordinate actions of digital circuits. Most integrated circuits (ICs) of sufficient complexity use a clock signal in order to synchronize different parts of the circuit, cycling at a rate slower than the worst-case internal propagation delays. The preeminent example of the aforementioned integrated circuit is the CPU 520, the central component of modern computers, which relies on a clock. The only exceptions are asynchronous circuits such as asynchronous CPUs. The clock 510 can comprise a plurality of embodiments, such as, but not limited to, single-phase clock which transmits all clock signals on effectively 1 wire, two- phase clock which distributes clock signals on two wires, each with non-overlapping pulses, and four-phase clock which distributes clock signals on 5 wires.

Many computing devices 500 use a “clock multiplier” which multiplies a lower frequency external clock to the appropriate clock rate of the CPU 520. This allows the CPU 520 to operate at a much higher frequency than the rest of the computer, which affords performance gains in situations where the CPU 520 does not need to wait on an external factor (like memory 550 or input/output 560). Some embodiments of the clock 510 may include dynamic frequency change, where, the time between clock edges can vary widely from one edge to the next and back again.

A system consistent with an embodiment of the disclosure the computing device 500 may include the CPU unit 520 comprising at least one CPU Core 521. A plurality of CPU cores 521 may comprise identical CPU cores 521, such as, but not limited to, homogeneous multi-core systems. It is also possible for the plurality of CPU cores 521 to comprise different CPU cores 521, such as, but not limited to, heterogeneous multi-core systems, big.LITTLE systems and some AMD accelerated processing units (APU). The CPU unit 520 reads and executes program instructions which may be used across many application domains, for example, but not limited to, general purpose computing, embedded computing, network computing, digital signal processing (DSP), and graphics processing (GPU). The CPU unit 520 may run multiple instructions on separate CPU cores 521 at the same time. The CPU unit 520 may be integrated into at least one of a single integrated circuit die and multiple dies in a single chip package. The single integrated circuit die and multiple dies in a single chip package may contain a plurality of other aspects of the computing device 500, for example, but not limited to, the clock 510, the CPU 520, the bus 530, the memory 550, and I/0 560.

The CPU unit 520 may contain cache 522 such as, but not limited to, a level 1 cache, level 2 cache, level 3 cache or combination thereof. The aforementioned cache 522 may or may not be shared amongst a plurality of CPU cores 521. The cache 522 sharing comprises at least one of message passing and inter-core communication methods may be used for the at least one CPU Core 521 to communicate with the cache 522. The inter-core communication methods may comprise, but not limited to, bus, ring, two-dimensional mesh, and crossbar. The aforementioned CPU unit 520 may employ symmetric multiprocessing (SMP) design.

The plurality of the aforementioned CPU cores 521 may comprise soft microprocessor cores on a single field programmable gate array (FPGA), such as semiconductor intellectual property cores (IP Core). The plurality of CPU cores 521 architecture may be based on at least one of, but not limited to, Complex instruction set computing (CISC), Zero instruction set computing (ZISC), and Reduced instruction set computing (RISC). At least one of the performance-enhancing methods may be employed by the plurality of the CPU cores 521, for example, but not limited to Instruction-level parallelism (ILP) such as, but not limited to, superscalar pipelining, and Thread-level parallelism (TLP).

Consistent with the embodiments of the present disclosure, the aforementioned computing device 500 may employ a communication system that transfers data between components inside the aforementioned computing device 500, and/or the plurality of computing devices 500. The aforementioned communication system will be known to a person having ordinary skill in the art as a bus 530. The bus 530 may embody internal and/or external plurality of hardware and software components, for example, but not limited to a wire, optical fiber, communication protocols, and any physical arrangement that provides the same logical function as a parallel electrical bus. The bus 530 may comprise at least one of, but not limited to a parallel bus, wherein the parallel bus carry data words in parallel on multiple wires, and a serial bus, wherein the serial bus carry data in bit-serial form. The bus 530 may embody a plurality of topologies, for example, but not limited to, a multidrop/electrical parallel topology, a daisy chain topology, and a connected by switched hubs, such as USB bus. The bus 530 may comprise a plurality of embodiments, for example, but not limited to:
Internal data bus (data bus) 531/Memory bus
Control bus 532
Address bus 533
System Management Bus (SMBus)
Front-Side-Bus (FSB)
External Bus Interface (EBI)
Local bus
Expansion bus
Lightning bus
Controller Area Network (CAN bus)
Camera Link
ExpressCard
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS)
HyperTransport
InfiniBand
RapidIO
Mobile Industry Processor Interface (MIPI)
Coherent Processor Interface (CAPI)
Plug-n-play
1-Wire
Peripheral Component Interconnect (PCI), including embodiments such as, but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCle v2], PCI Express External Cabling [ePCle], and PCI Express OCuLink [Optical Copper {Cu} Link]), Express Card, AdvancedTCA, AMC, Universal IO, Thunderbolt/Mini DisplayPort, Mobile PCIe (M-PCIe), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/105 bus (e.g., PC/105-Plus, PCI/105-Express, PCI/105, and PCI-105), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1395 Interface/Firewire, Thunderbolt, and extensible Host Controller Interface (xHCI).

Consistent with the embodiments of the present disclosure, the aforementioned computing device 500 may employ hardware integrated circuits that store information for immediate use in the computing device 500, know to the person having ordinary skill in the art as primary storage or memory 550. The memory 550 operates at high speed, distinguishing it from the non-volatile storage sub-module 561, which may be referred to as secondary or tertiary storage, which provides slow-to-access information but offers higher capacities at lower cost. The contents contained in memory 550, may be transferred to secondary storage via techniques such as, but not limited to, virtual memory and swap. The memory 550 may be associated with addressable semiconductor memory, such as integrated circuits consisting of silicon-based transistors, used for example as primary storage but also other purposes in the computing device 500. The memory 550 may comprise a plurality of embodiments, such as, but not limited to volatile memory, non-volatile memory, and semi-volatile memory. It should be understood by a person having ordinary skill in the art that the ensuing are non-limiting examples of the aforementioned memory:
Volatile memory which requires power to maintain stored information, for example, but not limited to, Dynamic Random-Access Memory (DRAM) 551, Static Random-Access Memory (SRAM) 552, CPU Cache memory 525, Advanced Random-Access Memory (A-RAM), and other types of primary storage such as Random-Access Memory (RAM).
Non-volatile memory which can retain stored information even after power is removed, for example, but not limited to, Read-Only Memory (ROM) 553, Programmable ROM (PROM) 555, Erasable PROM (EPROM) 555, Electrically Erasable PROM (EEPROM) 556 (e.g., flash memory and Electrically Alterable PROM [EAPROM]), Mask ROM (MROM), One Time Programable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory which may have some limited non-volatile duration after power is removed but loses data after said duration has passed. Semi-volatile memory provides high performance, durability, and other valuable characteristics typically associated with volatile memory, while providing some benefits of true non-volatile memory. The semi-volatile memory may comprise volatile and non-volatile memory and/or volatile memory with battery to provide power after power is removed. The semi-volatile memory may comprise, but not limited to spin-transfer torque RAM (STT-RAM).
Consistent with the embodiments of the present disclosure, the aforementioned computing device 500 may employ the communication system between an information processing system, such as the computing device 500, and the outside world, for example, but not limited to, human, environment, and another computing device 500. The aforementioned communication system will be known to a person having ordinary skill in the art as I/0 560. The I/O module 560 regulates a plurality of inputs and outputs with regard to the computing device 500, wherein the inputs are a plurality of signals and data received by the computing device 500, and the outputs are the plurality of signals and data sent from the computing device 500. The I/O module 560 interfaces a plurality of hardware, such as, but not limited to, non-volatile storage 561, communication devices 562, sensors 563, and peripherals 565. The plurality of hardware is used by the at least one of, but not limited to, human, environment, and another computing device 500 to communicate with the present computing device 500. The I/O module 560 may comprise a plurality of forms, for example, but not limited to channel I/O, port mapped I/O, asynchronous I/O, and Direct Memory Access (DMA).
Consistent with the embodiments of the present disclosure, the aforementioned computing device 500 may employ the non-volatile storage sub-module 561, which may be referred to by a person having ordinary skill in the art as one of secondary storage, external memory, tertiary storage, off-line storage, and auxiliary storage. The non-volatile storage sub-module 561 may not be accessed directly by the CPU 520 without using intermediate area in the memory 550. The non-volatile storage sub-module 561 does not lose data when power is removed and may be two orders of magnitude less costly than storage used in memory module, at the expense of speed and latency. The non-volatile storage sub-module 561 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (561) may comprise a plurality of embodiments, such as, but not limited to:
Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the aforementioned computing device 500 may employ the communication sub-module 562 as a subset of the I/0 560, which may be referred to by a person having ordinary skill in the art as at least one of, but not limited to, computer network, data network, and network. The network allows computing devices 500 to exchange data using connections, which may be known to a person having ordinary skill in the art as data links, between network nodes. The nodes comprise network computer devices 500 that originate, route, and terminate data. The nodes are identified by network addresses and can include a plurality of hosts consistent with the embodiments of a computing device 500. The aforementioned embodiments include, but not limited to personal computers, phones, servers, drones, and networking devices such as, but not limited to, hubs, switches, routers, modems, and firewalls.

Two nodes can be said are networked together, when one computing device 500 is able to exchange information with the other computing device 500, whether or not they have a direct connection with each other. The communication sub-module 562 supports a plurality of applications and services, such as, but not limited to World Wide Web (WWW), digital video and audio, shared use of application and storage computing devices 500, printers/scanners/fax machines, email/online chat/instant messaging, remote control, distributed computing, etc. The network may comprise a plurality of transmission mediums, such as, but not limited to conductive wire, fiber optics, and wireless. The network may comprise a plurality of communications protocols to organize network traffic, wherein application-specific communications protocols are layered, may be known to a person having ordinary skill in the art as carried as payload, over other more general communications protocols. The plurality of communications protocols may comprise, but not limited to, IEEE 802, ethernet, Wireless LAN (WLAN/Wi-Fi), Internet Protocol (IP) suite (e.g., TCP/IP, UDP, Internet Protocol version 5 [IPv5], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], and Integrated Digital Enhanced Network [IDEN]).

The communication sub-module 562 may comprise a plurality of size, topology, traffic control mechanism and organizational intent. The communication sub-module 562 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G,5G (such as WiMax and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line and wireless communications

The aforementioned network may comprise a plurality of layouts, such as, but not limited to, bus network such as ethernet, star network such as Wi-Fi, ring network, mesh network, fully connected network, and tree network. The network can be characterized by its physical capacity or its organizational purpose. Use of the network, including user authorization and access rights, differ accordingly. The characterization may include, but not limited to nanoscale network, Personal Area Network (PAN), Local Area Network (LAN), Home Area Network (HAN), Storage Area Network (SAN), Campus Area Network (CAN), backbone network, Metropolitan Area Network (MAN), Wide Area Network (WAN), enterprise private network, Virtual Private Network (VPN), and Global Area Network (GAN).

Consistent with the embodiments of the present disclosure, the aforementioned computing device 500 may employ the sensors sub-module 563 as a subset of the I/0 560. The sensors sub-module 563 comprises at least one of the devices, modules, and subsystems whose purpose is to detect events or changes in its environment and send the information to the computing device 500. Sensors are sensitive to the measured property, are not sensitive to any property not measured, but may be encountered in its application, and do not significantly influence the measured property. The sensors sub-module 563 may comprise a plurality of digital devices and analog devices, wherein if an analog device is used, an Analog to Digital (A-to-D) converter must be employed to interface the said device with the computing device 500. The sensors may be subject to a plurality of deviations that limit sensor accuracy. The sensors sub-module 563 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:
Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nano-sensors).
Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensor (guitar pickup), seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, bedwetting alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.
Navigation sensors, such as, but not limited to, air speed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as, but not limited to, accelerometer, displacement sensor, flex sensor, free fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, LiDAR, multi-spectral light sensor, colorimeter, contact image sensor, electro-optical sensor, infra-red sensor, kinetic inductance detector, LED as light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 500 may employ the peripherals sub-module 562 as a subset of the I/O 560. The peripheral sub-module 565 comprises ancillary devices uses to put information into and get information out of the computing device 500. There are 3 categories of devices comprising the peripheral sub-module 565, which exist based on their relationship with the computing device 500, input devices, output devices, and input/output devices. Input devices send at least one of data and instructions to the computing device 500. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to position of a mouse.
The number of degrees of freedom involved, such as, but not limited to, two-dimensional mice vs three-dimensional mice used for Computer-Aided Design (CAD) applications.

Output devices provide output from the computing device 500. Output devices convert electronically generated information into a form that can be presented to humans. Input/output devices perform that perform both input and output functions. It should be understood by a person having ordinary skill in the art that the ensuing are non-limiting embodiments of the aforementioned peripheral sub-module 565:
Input Devices
Human Interface Devices (HID), such as, but not limited to, pointing device (e.g., mouse, touchpad, joystick, touchscreen, game controller/gamepad, remote, light pen, light gun, Wii remote, jog dial, shuttle, and knob), keyboard, graphics tablet, digital pen, gesture recognition devices, magnetic ink character recognition, Sip-and-Puff (SNP) device, and Language Acquisition Device (LAD).
High degree of freedom devices, that require up to six degrees of freedom such as, but not limited to, camera gimbals, Cave Automatic Virtual Environment (CAVE), and virtual reality systems.
Video Input devices are used to digitize images or video from the outside world into the computing device 500. The information can be stored in a multitude of formats depending on the user's requirement. Examples of types of video input devices include, but not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
Audio input devices are used to capture sound. In some cases, an audio output device can be used as an input device, in order to capture produced sound. Audio input devices allow a user to send audio signals to the computing device 500 for at least one of processing, recording, and carrying out commands. Devices such as microphones allow users to speak to the computer in order to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.
Data Acquisition (DAQ) devices convert at least one of analog signals and physical parameters to digital values for processing by the computing device 500. Examples of DAQ devices may include, but not limited to, Analog to Digital Converter (ADC), data logger, signal conditioning circuitry, multiplexer, and Time to Digital Converter (TDC).

Output Devices may further comprise, but not be limited to:
Display devices, which convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).

Printers, such as, but not limited to, inkjet printers, laser printers, 3D printers, solid ink printers and plotters.
Audio and Video (AV) devices, such as, but not limited to, speakers, headphones, amplifiers and lights, which include lamps, strobes, DJ lighting, stage lighting, architectural lighting, special effect lighting, and lasers.
Other devices such as Digital to Analog Converter (DAC)

Input/Output Devices may further comprise, but not be limited to, touchscreens, networking device (e.g., devices disclosed in network 562 sub-module), data storage device (non-volatile storage 561), facsimile (FAX), and graphics/sound cards.

All rights including copyrights in the code included herein are vested in and the property of the Applicant. The Applicant retains and reserves all rights in the code included herein, and grants permission to reproduce the material only in connection with reproduction of the granted patent and for no other purpose.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A system for an automated medical data processing based on patient-related data, comprising:
a processor of a healthcare processing server node configured to host at least Artificial Intelligence (AI) and machine learning (ML) modules and connected to at least one medical records cloud-based database and to at least one patient entity node over a network; and
a memory on which are stored machine-readable instructions that when executed by the processor, cause the processor to:
acquire user account creation and input data from at least one patient entity node using an OCR module;
analyze patient intake data derived from the input data by an AI module configured to analyze the intake data;
process user insurance data by an insurance AI module configured to generate an insurance verification verdict;
acquire recommended lab test and triage data of the user and ingest the lab test and the triage data into an AI module configured to analyze the lab tests;
receive treatment and medication suggestions and generate a feature vector based on the treatment and medication suggestions;
provide the feature vector to an ML module configured to generate at least one clinical outcome model;
derive clinical documentation data from the patient intake data and from the at least one medical records cloud-based database and apply NPL processing to the clinical documentation data; and
acquire revenue cycle data from the clinical documentation data and ingest the revenue cycle data into an AI module configured to generate billing parameters.

2. The machine-readable instructions of claim 1 that when executed by the processor, cause the processor to generate personalized health insights for the patient based on outputs of the at least one clinical outcome model.

3. The machine-readable instructions of claim 1 that when executed by the processor, cause the processor to perform predictive analytics by any of the AI modules based on the clinical documentation data derived from the patient intake data and from the at least one medical records cloud-based database.

4. The machine-readable instructions of claim 1 that when executed by the processor, cause the processor to combine data received from the AI modules and to convert the data into at least one standardized format for data sharing.

5. The machine-readable instructions of claim 1 that when executed by the processor, cause the processor to onboard the healthcare processing server node and the at least one patient entity node onto a secured network.

6. The machine-readable instructions of claim 5 that when executed by the processor, cause the processor to execute at least one API call to record the clinical documentation data on a central secured database.

7. The machine-readable instructions of claim 6 that when executed by the processor, cause the processor to record the input data from the at least one patient entity node on the central secured database as an image-based file.

8. The machine-readable instructions of claim 7 that when executed by the processor, cause the processor to record patient interaction logs and the personalized health insights corresponding to the image-based file on the central secured database.

9. The machine-readable instructions of claim 7 that when executed by the processor, cause the processor to record outputs of the AI modules and the at least one clinical outcome model corresponding to the image-based file on the central secured database.

10. The machine-readable instructions of claim 7 that when executed by the processor, cause the processor to, responsive to receiving updated input data from the at least one patient entity node, generate a new image-based file corresponding to the at least one patient entity.

11. A method for an automated medical data processing based on patient-related data, comprising:
acquiring, by a healthcare processing server (HPS) node configured to host at least Artificial Intelligence (AI) and machine learning (ML) modules, a user account creation and input data from at least one patient entity node using an OCR module;
analyzing, by the HPS node, patient intake data derived from the input data by an AI module configured to analyze the intake data;
processing, by the HPS node, user insurance data by an insurance AI module configured to generate an insurance verification verdict;
acquiring, by the HPS node, recommended lab test and triage data of the user and ingesting the lab test and the triage data into an AI module configured to analyze the lab tests;
receiving, by the HPS node, treatment and medication suggestions and generating a feature vector based on the treatment and medication suggestions;
providing, by the HPS node, the feature vector to an ML module configured to generate at least one clinical outcome model;
deriving, by the HPS node, clinical documentation data from the patient intake data and from the at least one medical records cloud-based database and applying NPL processing to the clinical documentation data; and
acquiring, by the HPS node, revenue cycle data from the clinical documentation data and ingesting the revenue cycle data into an AI module configured to generate billing parameters.

12. The method of claim 11, further comprising generating personalized health insights for the patient based on outputs of the at least one clinical outcome model.

13. The method of claim 11, further comprising performing predictive analytics by any of the AI modules based on the clinical documentation data derived from the patient intake data and from the at least one medical records cloud-based database.

14. The method of claim 11, further comprising combining data received from the AI modules and to converting the data into at least one standardized format for data sharing.

15. The method of claim 11, further comprising onboarding the healthcare processing server node and the at least one patient entity node onto a secured network.

16. The method of claim 15, further comprising execute at least one API call to record the clinical documentation data on a central secured database.

17. The method of claim 15, further comprising recording the input data from the at least one patient entity node on the central secured database as an image-based file.

18. A non-transitory computer-readable medium comprising instructions, that when read by a processor, cause the processor to perform:
acquiring a user account creation and input data from at least one patient entity node using an OCR module;
analyzing patient intake data derived from the input data by an AI module configured to analyze the intake data;
processing user insurance data by an insurance AI module configured to generate an insurance verification verdict;
acquiring recommended lab test and triage data of the user and ingesting the lab test and the triage data into an AI module configured to analyze the lab tests;
receiving treatment and medication suggestions and generating a feature vector based on the treatment and medication suggestions;
providing the feature vector to an ML module configured to generate at least one clinical outcome model;
deriving clinical documentation data from the patient intake data and from the at least one medical records cloud-based database and applying NPL processing to the clinical documentation data; and
acquiring revenue cycle data from the clinical documentation data and ingesting the revenue cycle data into an AI module configured to generate billing parameters.

19. The non-transitory computer readable medium of claim 18, further comprising instructions, that when read by the processor, cause the processor to perform predictive analytics by any of the AI modules based on the clinical documentation data derived from the patient intake data and from the at least one medical records cloud-based database.

20. The non-transitory computer readable medium of claim 18, further comprising instructions, that when read by the processor, cause the processor to generating personalized health insights for the patient based on outputs of the at least one clinical outcome model.
